(19) United States
(12) Patent Application Publication
(10) Pub. No.: US 2025/0049560 A1
(43) Pub. Date: Feb. 13, 2025

(54) Implant System and Method for Producing an Implant System

(71) Applicant: Karl Leibinger Asset Management GmbH & Co. KG, Mühlheim an der Donau (DE)

(72) Inventors: Stefanie Grom, Neuhausen ob Eck (DE); Frank Reinauer, Emmingen-Liptingen (DE); Tobias Wolfram, Dreieich (DE)

(21) Appl. No.: 18/722,401

(22) PCT Filed: Nov. 30, 2022
(86) PCT No.: PCT/EP2022/083763

§ 371 Date: Jun. 20, 2024

(30) Foreign Application Priority Data
Dec. 22, 2021 (DE) 10 2021 214 856.5

(51) Int. Cl. A61F 2/12; A61F 2/00; A61L 27/04; A61L 27/10; A61L 27/24; A61L 27/26; A61L 27/54; A61L 27/58

(52) U.S. Cl. CPC A61F 2/12; A61F 2/0059; A61F 2/0077; A61L 27/04; A61L 27/10; A61L 27/24; A61L 27/26; A61L 27/54; A61L 27/58; A61F 2002/0086; A61F 2210/0004; A61F 2220/0008; A61F 2230/0063; A61F 2240/002; A61F 2250/0024

(57) ABSTRACT

The invention relates to an implant system, comprising at least one bioresorbable enveloping element which is designed with passage openings and which defines a three-dimensional interior for receiving soft tissue. Furthermore, the implant system can have an inner element which is or can be arranged at least partially in the three-dimensional interior defined by the enveloping element and which has at least one guide structure for a cell migration. Moreover, the invention relates to a method for producing an implant system.

### TECHNICAL FIELD

The present invention relates to an implant system and to a method for producing implant systems. The implant system may, in particular, be a resorbable geometrically stable implant system for the augmentation of large-volume soft tissue defects or for breast reconstruction.

### BACKGROUND OF THE INVENTION

Conventional implants for the reconstruction of large-volume soft tissue defects (for example breast reconstructions) or for the augmentation of large soft tissue defects are not biodegradable and therefore remain as a foreign body in the region of the patient. Bioresorbable implants comprising xenogenic material have hitherto been possible only for very small defects, such implants often having an unfavorably small ratio of volume to mass, that is to say they are excessively heavy for a desired volume. Rejection reactions or inflammatory reactions may lead to immunological complications. Such an effect may be exacerbated by aging of the implant material if it is not bioresorbable. Such aging may also lead to leakage of the implant, which may sometimes necessitate revision of the implant.

### SUMMARY OF THE INVENTION

It is an object of the present invention to provide an improved implant system for the reconstruction of soft tissue defects or for the augmentation of soft tissue defects, which solves the aforementioned problems or at least mitigates their consequences. This object is achieved by an implant system having the features of patent claim 1. It is furthermore an object to provide a method for producing such an implant system. This object is achieved by a method having the features of patent claim 12.

Correspondingly, according to a first aspect of the present invention, an implant system is provided which comprises at least one bioresorbable capsule element, which is configured with passage openings and defines a three-dimensional interior for receiving soft tissue. The soft tissue may, for example, be adipose tissue and/or connective tissue, in particular for breast reconstruction or breast augmentation.

The implant system is preferably intended for use on mammals, above all on human patients. Whenever “patient” or “a patient” is referred to here and in what follows, this is meant to include both male and female patients. In particular, female patients are meant when implants for breast reconstruction are involved. In fact, an implant for breast reconstruction will be described herein as a common example.

In connection with the present description, “bioresorbable” is intended to mean that the corresponding bioresorbable element, here the capsule element, is not only tolerated very well by the body into which it is implanted, but is also gradually resorbed by the latter. In particular, this has the advantage that the implant does not necessarily need to be removed again at a later time but will have been fully resorbed and/or for instance replaced by endogenous regrown tissue. The terms “bioresorbable” and “biodegradable” will both be used below. “Bioresorbable” essentially means physiological absorption of the breakdown products by cells, while “biodegradable” refers to primarily extracellular breakdown without physiological incorporation of the breakdown products. In the context of the present invention, the two terms are therefore interchangeable insofar as an element described as “bioresorbable” may also (as an alternative or in addition) be configured to be “biodegradable”, and vice versa.

The defined three-dimensional interior may have any desired shape, for example a hemispherical shape or the shape of a segment of a sphere. Other shapes may however also be envisioned, depending on which soft tissue is intended to be received at which position and in which body. The purpose of the implant system is in this case, in particular, to receive the soft tissue, generate cavities with stable volumes and fix them in position at least while a healing process and a gradual resorption of the capsule element is taking place. As will be explained in more detail below, the capsule element may fulfill further tasks, for example promoting or stimulating tissue regeneration. This may also be further promoted by other elements and properties of the implant system. In this case, in particular, site-specific inward cell migration may be made possible by specific implant embodiments in combination with membrane barriers and the inward cell migration may be controlled. The membranes that form the membrane barriers are specifically fixable or fixed on defined implant structures.

The capsule element is so named because, in particular while it is being used in the body of a patient, it encapsulates (fully or at least partially) the soft tissue received in the interior, and therefore not only fixes it but protects and possibly even isolates it from external effects. The passage openings in the capsule element are used to exchange substances between the interior and the exterior which bears on the other side of the capsule element, and which likewise still lies inside the body of the patient. In this way, for example, cells may migrate inward, fluids may be exchanged, blood vessels may grow through and/or the like. The size or sizes of the passage openings may correspondingly be adapted to the desired function and to the substances and/or tissue parts to be exchanged. In addition, the implant system, in particular the capsule element, may be covered with a membrane in order to act as a diffusion barrier. The membrane may in this case have a different degradation profile than the actual implant system. This prevents inflammatory cells from migrating inward at an early point in time. Only after degradation of the membrane can tissue-specific cells then migrate inward, after the primary inflammatory reaction has subsided.

The capsule element is preferably configured to be geometrically stable, i.e. insofar as it loses its shape only (at most) gradually and in a defined way in the course of the progressive biodegradation and bioresorption by the body into which it is implanted, but advantageously substantially not by forces which act on the capsule element from the start in the implant situation. In this way, the implant system according to the invention differs from so-called “injectables”, which have no geometrical stability even at the start of the implantation. A geometrical stability against thermal deformation up to at least 60° C. is likewise preferred.

According to a second aspect, the invention provides a method for producing an implant system, comprising the steps:
measuring the anatomy of a human patient in order to obtain anatomical data; and
creating a patient-specific implant system on the basis of the anatomical data.

The capsule element of the implant system may preferably be produced by thermoforming (sometimes also known as “deep drawing”). Passage openings may be introduced in a defined way directly during the deep drawing (as “primary passage openings”) and/or subsequently (as “secondary passage openings”) by means of subtractive methods. The inner element may also be created by additive manufacturing technologies, and may be connected or connectable to the capsule element on specific holding structures. In addition, both the inner element and the capsule element may be manufactured patient-specifically. The capsule element and/or the inner element may consist either of the same raw material or of a different material combination, which may for example ensure that the interior can be colonized more rapidly by tissue and the capsule element remains geometrically stable for some time longer, until it is ultimately resorbed.

According to a third aspect, the invention provides a method of fixing a soft tissue in the body of a patient, comprising the steps:
fitting an implant system according to one embodiment of the first aspect of the present invention into the body of a patient;
moving the soft tissue and/or the implant system in such a way that the three-dimensional interior defined by the capsule element receives the soft tissue.

Depending on whether or not the soft tissue is already connected to the body of the patient, the soft tissue may be introduced into the implant system, that is to say into the interior defined by the capsule element, before the implant system is introduced into the body of the patient, or this may be carried out in the reverse order or simultaneously.

Advantageous and preferred embodiments, variants or refinements of embodiments will be explained in more detail in the dependent claims and the following description, in particular with reference to the figures.

According to some preferred embodiments, variants or refinements of embodiments, the three-dimensional interior is defined by a three-dimensional curvature of the capsule element and is at least partially enclosed thereby. If the three-dimensional interior is for example to be configured hemispherically, the capsule element may be formed in the shape of a hemispherical shell. The capsule element may enclose the three-dimensional interior fully, for example in order to ensure particular stability, or alternatively the capsule element may also enclose the three-dimensional interior only partially so that an inlet opening remains, through which for example the soft tissue may be introduced into the interior. In comparison with other options for defining an interior, a curvature is particularly natural and may thus be adapted ideally to the various tissue parts in the body of the patient, cause little irritation and feel natural.

According to some preferred embodiments, variants or refinements of embodiments, the bioresorbable capsule element is configured as a capsule element which is geometrically stable before the onset of bioresorption. In some variants, instead of this or in addition, the inner element may also be configured as a geometrically stable inner element, for example in order to impart additional stability to the implant system. The variation of the geometrical stability as a function of time due to bioresorption may be adjusted application- and patient-specifically by corresponding selection of materials and geometries.

According to some preferred embodiments, variants or refinements of embodiments, the passage openings are provided by pores and/or by a matrix grid. The pores and/or the matrix grid may in this case advantageously be technically minimized, that is to say in particular selected and configured so that, for the desired strength and stability of the capsule element, the amount of implant material used is minimized. In this way, for example, the ratio between a volume of the three-dimensional interior and a mass of the implant system, in particular of the capsule element, may advantageously be increased. The passage openings in the capsule element are therefore used not only for passage of substances or tissue parts, but also for material reduction and weight reduction of the capsule element.

The passage openings may also be arranged in site-specific patterns. For example, marginal, central or unilaterally oriented passage openings are possible. In addition, the resorption kinetics may be adjusted by means of the specific configuration of the passage openings.

If the passage openings are formed at least partially by pores, the pores may at least partially be arranged uniformly. That is to say, there may be at least one subregion of the capsule element in which the pores are arranged at regular distances or in a regular pattern. In one or more other regions of the capsule element, on the other hand, the pores may be arranged more densely or spread out in relation to the pattern, in which case the corresponding locations may be coordinated with tissues to be arranged inside or outside the interior. In other words, where increased penetration of the capsule element by soft tissue or increased exchange of substances through the capsule element is desired, an arrangement of pores in a greater frequency and/or with larger holes may be provided than at a different location of the capsule element, where less exchange or less ingress is desired. In other words, both the distances between the pores and the dimensions, that is to say in particular the diameters, of the pores themselves may be different at various locations of the capsule element, that is to say they may vary over the capsule element. The same applies for a matrix grid of passage openings, in which case the passage openings may also be configured as squares or rectangles. Passage openings configured as pores with an elliptical or circular shape may also be arranged in a grid.

According to some preferred embodiments, variants or refinements of embodiments, the capsule element comprises a biodegradable polymer or consists of a biodegradable polymer. Advantageously, the capsule element may also comprise a biodegradable polymer composite or consist of a biodegradable polymer composite. In this case, it is particularly preferred for the capsule element to comprise a biodegradable polyester or consist of a biodegradable polyester, or comprise a biodegradable polyester composite or consist of a biodegradable polyester composite. It is particularly preferred for the capsule element to comprise or consist of one of the following biodegradable polymers (either pure or in the form of a composite):
poly (L-lactic acid), PLLA
poly (L-lactic acid)-glycolic acid, PLLA-PGA
poly (lactic acid-co-glycolic acid), LGA
polycaprolactone, PCL
poly (D-L-lactic acid), PDLLA.

These polymers are particularly suitable because of their biodegradability and because of the time delay in the biodegradation or bioresorption by the body of the patient.

The inner element may be formed from the same material as the capsule element or from a different material. For example, the capsule element may be formed from PDLLA and the inner element may be formed from PCL.

According to some preferred embodiments, variants or refinements of embodiments, the capsule element comprises ceramic particles, metal particles and/or bioglass particles. Such particles may, in particular, be part of various composites or compoundings of the capsule element. Such particles may advantageously promote and stimulate the tissue regeneration, and thereby increase a therapeutic usefulness of the implant system. The capsule element therefore also offers a highly suitable solution for administering such therapeutically useful particles precisely positioned and with a time delay into the body of the patient, without for example regular injection or the like being necessary therefor.

According to some preferred embodiments, variants or refinements of embodiments, the three-dimensional interior defined by the capsule element has an inlet opening, which is dimensioned in such a way that the soft tissue can be introduced into the interior through the inlet opening. The inlet opening is in this case, in particular, larger than the passage openings. For example, the inlet opening may have a diameter at the widest location which is more than 10 times as large as a diameter (in the case of circular or elliptical pores) or an edge length (in the case of square or rectangular passage openings) of the largest or smallest passage opening, in particular more than 15 times as large, more than 20 times as large or even larger. Preferably, the capsule element comprises a multiplicity of passage openings, for example at least 10, at least 30 passage openings, at least 50 passage openings, more than 100 passage openings or more.

On the other hand, it is preferred for the capsule element to comprise at most two inlet openings for the soft tissue, particularly preferably precisely one inlet opening for the soft tissue into the interior. The inlet opening also differs from the passage openings particularly in that when the implant system, more precisely the capsule element, is fitted, the soft tissue is preferably introduced through the inlet opening into the interior, while a passage of substances or tissue parts through the passage openings only occurs gradually after the implant system is fitted into the body of the patient.

According to some preferred embodiments, variants or refinements of embodiments, a holding structure for retaining the implant system, in particular the capsule element, in the body of a patient is formed at least partially on an edge of the inlet opening. The holding structure may, for example, be formed by a marginal shaping or eversion, which in some variants may be partially structured or specifically functionalized. The holding structure may also comprise one or more fixing elements, which may for example be configured in the form of nails or pinned for introduction into a hard tissue of the body of the patient or in the form of a loop for fixing of the capsule element on a hard tissue of the patient, or the like. In this way, the implant system may be retained particularly stably in the body of the patient.

According to some preferred embodiments, variants or refinements of embodiments, the implant system furthermore comprises an inner element, which is arranged or arrangeable at least partially in the three-dimensional interior defined by the capsule element. The inner element may fulfill a multiplicity of functions, and the inner element may for example comprise at least one guide structure for inward cell migration. It is also conceivable for the inner element to comprise at least one supporting structure for maintaining a three-dimensional envelope, for instance the three-dimensional curvature, of the capsule element in order to define the three-dimensional interior. For example, the inner element may comprise supporting walls or supporting columns comparable to the interior of a house, or stiffening elements such as ribs on the inner side (the side of the capsule element turned toward the interior) or the like. It is to be understood that stiffening elements, for example ribs, may also be arranged on the outer side of the capsule element.

The inner element may be configured integrally together with the capsule element, and may in particular be produced simultaneously therewith. As an alternative, the inner element may also be configured separately from the capsule element. In the latter case, the inner element may be releasably connected, non-releasably connected or unconnected to the capsule element. For example, the inner element may be fitted into the capsule element when the implant system is implanted into the body of the patient, and then fixed in position by the capsule element.

According to some preferred embodiments, variants or refinements of embodiments, the inner element comprises ceramic particles, metal particles and/or bioglass particles. Such particles may, in particular, be part of various composites or compoundings of the capsule element. Such particles may advantageously promote and stimulate the tissue regeneration, and thereby increase a therapeutic usefulness of the implant system. The particles may also be used as a carrier system for specific growth factors, for example FGF (fibroblast growth factors), IGF-1 (insulin-like growth factors) or CTGF (connected tissue growth factor), and peptide-based variants of these growth factors, and therefore create optimal soft tissue regeneration by biological activation at site-specific locations due to the spatial arrangement in the implant system.

The inner element therefore also offers a highly suitable solution for administering such therapeutically useful particles precisely positioned and with a time delay into the body of the patient, without for example regular injection or the like being necessary therefor. The inner element (or the inner structure) of the implant system may also be produced by specific incorporation of constituents of the extracellular matrix, for example collagen-based fiber-like components, although fibronectin or hyaluronic acid components would also be possible, in order to accelerate and/or improve tissue healing.

According to some preferred embodiments, variants or refinements of embodiments, the interior of the implant system is filled at least partially with a collagen matrix and/or a biocomposite mixture. This may additionally promote inward tissue migration.

### BRIEF DESCRIPTION OF THE FIGURES

The invention will be explained in more detail below with the aid of exemplary embodiments in the figures of the drawings. Sometimes in a schematic representation:

FIG. 1 shows an exemplary three-dimensional plan view of an implant system according to one embodiment of the present invention;

FIG. 2 shows a schematic cross-sectional view through the implant system according to FIG. 1 in one variant; and

FIG. 3 shows a schematic flowchart to explain a production method according to another embodiment of the present invention.

In all the figures, elements and devices which are the same or functionally equivalent—unless otherwise indicated—have been provided with the same reference signs.

### DETAILED DESCRIPTION OF THE FIGURES

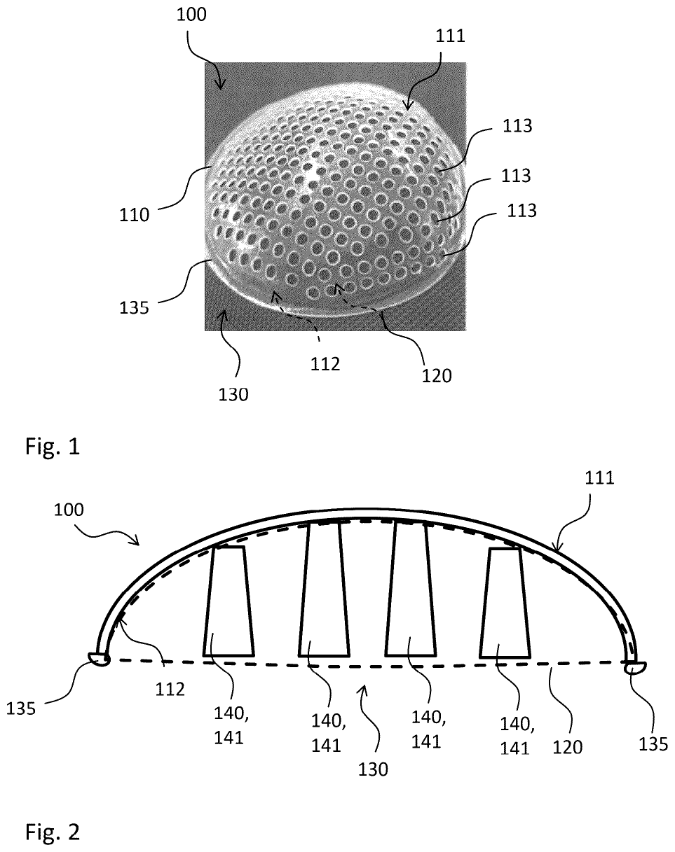

FIG. 1 shows an exemplary three-dimensional oblique view of an implant system 100 according to one embodiment of the present invention. The implant system 100 comprises a bioresorbable capsule element 110, which comprises a biodegradable or bioresorbable polymer or consists of a biodegradable or bioresorbable polymer. Preferably, the capsule element 110 comprises a biodegradable or bioresorbable polyester or consists of a biodegradable or bioresorbable polyester. Particularly suitable materials of which the capsule element 110 may consist, or which the capsule element 110 may comprise, are:
poly (L-lactic acid), PLLA
poly (L-lactic acid)-glycolic acid, PLLA-PGA
poly (lactic acid-co-glycolic acid), LGA
polycaprolactone, PCL
poly (D-L-lactic acid), PDLLA.

The capsule element 110 may preferably comprise ceramic particles, metal particles and/or bioglass particles, which may in particular promote and stimulate tissue regeneration and therefore have a therapeutic usefulness.

As may be seen in FIG. 1, the capsule element 110 has a three-dimensional curvature, that is to say it has a three-dimensional envelope without internally lying bends or edges. In the example shown in FIG. 1, the capsule element 110 is three-dimensionally curved in such a way that it has the shape of a spherical shell segment or, in other words, the shape of the spherical cap of a spherical segment. The capsule element 110 therefore essentially has two surfaces, a convex outwardly directed outer face 111 and an inwardly directed concave inner face 112. In FIG. 1, the outer face 111 is turned toward the observer while the inner face 112 is turned away from the observer.

On its inner side, that is to say in particular along the inner face 112, the capsule element 110 defines a three-dimensional interior 120 for receiving a soft tissue. The soft tissue may, for example, be adipose tissue and/or connective tissue. Because of the aforementioned shape of the capsule element 110, the interior 120 defined by the capsule element 110 has the shape of a spherical segment. Owing to the fact that the capsule element 110 is configured in the shape of a spherical shell segment, the three-dimensional interior 120 is partially open outward, that is to say there is an inlet opening 130. The inlet opening 130 is dimensioned in such a way that the soft tissue can be introduced through the inlet opening 130 into the interior 120. As a result of the geometry shown in FIG. 1 and mentioned above, the inlet opening 130 of the capsule element 110 is in this case circular.

The implant system 100 according to FIG. 1 may, for example, be used as a soft tissue implant for a breast reconstruction, the curved shape of the capsule element 110 being suitable for fixing the soft tissue received in the interior 120, bringing it into a shape suitable for the breast reconstruction, and keeping it in this shape. It is to be understood that other, for example less regular, shapes of the capsule element 110, and therefore also of the interior 120 thereby defined and the correspondingly defined inlet opening 130, are possible for soft tissue reconstructions or soft tissue augmentations on other parts of the body.

As is likewise shown in FIG. 1, the capsule element 110 preferably comprises a multiplicity of passage openings 113, which cross the capsule element 110 from the outer face 111 to the inner face 112. As already described above, these passage openings 113 are advantageously not only used to reduce the mass of the capsule element 110 while simultaneously maintaining the volume of the interior 120 defined by the capsule element 110. In addition, the passage openings 113 are also used to allow the exchange of tissue parts or other substances, for example fluids, through the capsule element 110. In this way, for instance, after the implant system 100 has been fitted into the body of a patient, blood vessels located on the outer face 111 of the capsule element 110 may grow into the capsule element 110. Furthermore, for example, lymph fluids may be exchanged, and/or the like. During the exchange of substances, ceramic particles, metal particles and/or bioglass particles contained in the capsule element 110 may advantageously be dissolved and therefore introduced into the interior 120 and the soft tissue located therein. In this way, the inward tissue migration or inward cell migration may be further improved.

As is shown in FIG. 1, the passage openings 113 of the capsule element 110 in the example shown are all configured with the same diameter and are arranged for the most part in a uniform grid structure or matrix grid, which is merely interrupted somewhat in the edge regions of the outer face 111. It is, however, to be understood that the passage openings 113 may also be site-specifically configured or arranged differently, for example in order to construct a diffusion barrier at particular sites in the tissue or to minimize such a diffusion barrier, or to facilitate diffusion at defined sites. In order to reduce the diffusion barrier, the passage openings 113 may be configured with a larger diameter or they may be formed in a higher density with respect to one another and formed in a larger number, while in order to construct a diffusion barrier or reinforce a diffusion barrier precisely the opposite measures may be taken, except for sections of the capsule element 110 in which no passage openings 113 are arranged. In FIG. 1, for example, it may be seen that regions without passage openings 113 may sometimes be formed close to the edge of the outer face 111.

It may furthermore be seen in FIG. 1 that the outer face 111 may be turned outward, that is to say in the direction of the outer face 111, marginally, that is to say on its radial circumference. In this way, a holding structure 135 for retaining the implant system 100, in particular the capsule element 110, in the body of the patient is formed on the capsule element 110.

FIG. 2 shows a schematic cross-sectional view through the implant system 100 according to FIG. 1 in one variant.

FIG. 2 clearly shows the marginal eversion which forms the holding structure 135, and which in this case extends entirely around the inlet opening 130. The passage openings 113 are not indicated in FIG. 2 for the sake of clarity. In the variant represented in FIG. 2, the implant system 100 additionally comprises a schematically represented inner element 140, which is arranged or arrangeable at least partially in the three-dimensional interior 120 defined by the capsule element 110 and which comprises at least one guide structure 141 for inward cell migration.

As already mentioned in detail above, the inner element 140 may be configured integrally together with the capsule element 110 or separately from the capsule element 110. The inner element 140 may be fastened on the capsule element 110 or fastenable thereon, or configured separately therefrom and loosely. The inner element 140 itself may be configured in one piece or consist of various individual elements. The inner element 140 may be configured from the same material as the capsule element 110 or from a different material, in which case the same materials, material composites or material combinations as described in detail above for the capsule element 110 may in principle be envisioned for the inner element 140. In particular, the inner element 140 may also comprise ceramic particles, metal particles and/or bioglass particles in order to promote and stimulate the tissue regeneration and thereby provide an additional therapeutic usefulness.

As is indicated in FIG. 2, the individual parts of the inner element 140 may not only function as a guide structure 141, but may optionally also function as a supporting structure or supporting structures for maintaining the three-dimensional envelope of the capsule element 110, that is to say for maintaining the curvature of the capsule element 110.

In FIG. 2, the guide structures 141 are represented as being relatively coarsely configured, although it is to be understood that they may also be formed with arbitrarily intricate and fine structures. The inner element 140 and/or the individual parts of the inner element 140, for example in particular any supporting structures of the inner element 140, may be configured differently to the capsule element 110 in respect of their biodegradability as a function of time. In this case, the various chronological sequences of the biodegradation may be adapted specially to the planned intended use, and for example the capsule element 110 may be intended to degrade before the inner element 140, or vice versa. It may also be intended that individual parts or sections of the inner element 140 degrade at different rates, for example in order to gradually provide volume in the interior 120 for the inward tissue migration, while in its turn the ordering or supporting function of the inner element 140 may be reduced by the inward tissue migration. The interior 120 may be filled at least partially or entirely with a collagen matrix and/or a biocomposite mixture, in order to further assist inward cell migration or inward tissue migration.

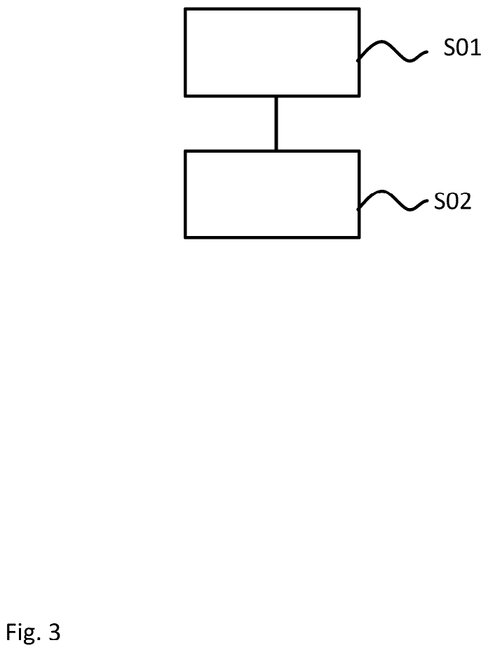

FIG. 3 shows a schematic flowchart to explain a method for producing an implant system 100 according to another embodiment of the present invention. The method according to FIG. 3 may be used to produce the implant system 100 shown in FIG. 1 or FIG. 2, and may correspondingly be adapted and/or varied according to all in relation to the implant system according to the invention, in particular according to FIG. 1 and FIG. 2, and vice versa.

In a step S10, the anatomy of a human patient is measured in order to obtain anatomical data. The anatomical data may, for example, involve the thickness and position of one or more types of tissue, the dimensions of a soft tissue defect and/or the like. In a step S20, a patient-specific implant system 100 is created on the basis of the anatomical data, that is to say initially planned and then produced according to the plan. The capsule element 110 of the implant system 100 may, in particular, be produced by thermoforming. An optionally provided inner element 140, which is not configured integrally with the capsule element 110, may either be connected to the capsule element 110 before the implantation and then implanted together therewith, or only fitted into the body of the patient and then connected in situ to the capsule element 110.

Although the present invention has been described above with the aid of preferred exemplary embodiments, it is not restricted thereto but may be modified in a variety of ways. In particular, the invention may be altered or modified in many ways without departing from the essential nature of the invention.

### LIST OF REFERENCE SIGNS

100 implant system
110 capsule element
111 outer face
112 inner face
113 passage openings
120 interior
130 inlet opening
135 holding structure
140 inner element
141 guide structure

1. An implant system comprising at least one bioresorbable patient-specific capsule element that is configured with passage openings and defines a three-dimensional interior for receiving soft tissue, wherein the three-dimensional interior defined by the capsule element has an inlet opening that is dimensioned in such a way that the soft tissue can be introduced into the interior through the inlet opening, and wherein a holding structure for retaining the implant system in the body of a patient is formed at least partially on an edge of the inlet opening.

2. The implant system as claimed in claim 1,
wherein the bioresorbable capsule element is configured as a capsule element that is geometrically stable before the onset of bioresorption.

3. The implant system as claimed in claim 1, wherein the three-dimensional interior is defined and at least partially enclosed by a three-dimensional curvature of the capsule element.

4. The implant system as claimed in claim 1, wherein the passage openings are provided by pores and/or by a matrix grid.

5. The implant system as claimed in claim 4, wherein the pores and/or the matrix grid are at least partially arranged or configured uniformly.

6. The implant system as claimed in claim 1, wherein the capsule element comprises a biodegradable polyester.

7. The implant system as claimed in claim 6, wherein the biodegradable polyester is selected from PLLA, PLLA-PGA, PLGA, PCL and PDLLA.

8. The implant system as claimed in claim 1, wherein the capsule element comprises ceramic particles, metal particles and/or bioglass particles.

9. (canceled)

10. (canceled)

11. The implant system (100) as claimed in claim 1, additionally comprising an inner element that is arranged or arrangeable at least partially in the three-dimensional interior defined by the capsule element and that comprises at least one guide structure for inward cell migration.

12. The implant system as claimed in claim 11, wherein the inner element has at least one supporting structure for maintaining a three-dimensional envelope of the capsule element in order to define the three-dimensional interior.

13. The implant system as claimed in claim 11, wherein the inner element is configured integrally with the capsule element.

14. The implant system as claimed in of claim 11, wherein the inner element comprises ceramic particles, metal particles and/or bioglass particles.

15. The implant system as claimed in claim 14, wherein the ceramic particles, metal particles and/or bioglass particles are used as a carrier system for growth factors.

16. The implant system as claimed in of claim 1, wherein the interior is filled at least partially with a collagen matrix and/or a biocomposite mixture.

17. A method for producing an implant system, comprising the steps of:
measuring the anatomy of a human patient in order to obtain anatomical data;
creating a patient-specific implant system on the basis of the anatomical data, the capsule element of the implant system being produced by thermoforming.

18. The implant system as claimed in claim 11, wherein the inner element is configured separately from the capsule element.

19. The implant system of claim 15, wherein the growth factors include one or more of FGF, IGF-1 or CTGF, or peptide-based variants of thereof.
